SEQUENCE LISTING (10 sequences)

SEQ ID NO: 1 - DNA, 18 bp, Artificial Sequence
  PCR PRIMER
aggaccgcgtgaccacct

SEQ ID NO: 2 - DNA, 16 bp, Artificial Sequence
  PCR PRIMER
cgtccagaatgccgga

SEQ ID NO: 3 - DNA, 20 bp, Artificial Sequence
  PCR PRIMER
cgactttgcagagatgtcca

SEQ ID NO: 4 - DNA, 20 bp, Artificial Sequence
  PCR PRIMER
catccacagagcgatgttgt

SEQ ID NO: 5 - DNA, 20 bp, Artificial Sequence
  PCR PRIMER
tgcagaggatgattgctgac

SEQ ID NO: 6 - DNA, 20 bp, Artificial Sequence
  PCR PRIMER
ggaggaagtccagtgtccag

SEQ ID NO: 7 - DNA, 20 bp, Artificial Sequence
  PCR PRIMER
gtctacgtcccgccataaaa

SEQ ID NO: 8 - DNA, 20 bp, Artificial Sequence
  PCR PRIMER
aggcagtgaagggactagca

SEQ ID NO: 9 - DNA, 20 bp, Artificial Sequence
  PCR PRIMER
gtaacccgttgaaccccatt

SEQ ID NO: 10 - DNA, 20 bp, Artificial Sequence
  PCR PRIMER
ccatccaatcggtagtagcg

(19) United States
(12) Patent Application Publication
(10) Pub. No.: US 2010/0298434 A1
(43) Pub. Date: Nov. 25, 2010

(54) NEUROPROTECTION AND PREVENTION OF DOPAMINERGIC CELL DEATH BY TARGETING NUR77 TRANSLOCATION

(76) Inventors: CLAUDE ROUILLARD, QUEBEC (CA); DANIEL LÉVESQUE, SAINTE-ANNE-DE-BELLEVUE (CA); MARIA-GRAZIA MARTINOLI, TROIS-RIVIERES (CA)

Correspondence Address: OGILVY RENAULT LLP, 1, Place Ville Marie, SUITE 2500, MONTREAL, QC, H3B 1R1, CA

(21) Appl. No.: 12/783,660

(22) Filed: May 20, 2010

Related U.S. Application Data
(60) Provisional application No. 61/180,198, filed on May 21, 2009.

(51) Int. Cl. A61K 31/202; A61P 25/16; C12Q 1/68; C12Q 1/02; G01N 33/53; C12N 5/02

(52) U.S. Cl. 514/560; 435/6; 435/29; 435/7.1; 435/375

(57) ABSTRACT

There is provided methods for the prevention of dopaminergic cell death for neuroprotection and the prevention or slow down of progression of Parkinson's disease. Particularly, the invention relates to methods for protecting and/or preventing dopaminergic cell death by the administration of a compound or agent inhibiting the translocation of Nur77 to the cytosol of dopaminergic cells. The invention also relates to methods for identifying such compounds or agents.

This application claims priority from U.S. provisional patent application 61/180,198 filed on May 21, 2009.

### FIELD OF THE INVENTION

The present invention relates to prevention or inhibition of dopaminergic cell death for neuroprotection and the protection, prevention, reduction of symptoms, delaying progression or treatment of Parkinson's disease. Particularly, the invention relates to methods for protecting and/or preventing dopaminergic cell death by administering to a subject in need thereof a compound, agent or composition inhibiting the translocation of Nur77 to the cytosol of dopaminergic cells. The invention also relates to methods for identifying such compounds or agents.

### BACKGROUND OF THE INVENTION

Parkinson's disease (PD) is a slow progressive neurodegenerative disorder. Most cases of PD are sporadic but rare familial forms of the disease do exist. The pathological hallmark of PD is a striking loss of dopamine (DA)-producing neurons in the substantia nigra (SN) causing DA depletion in the striatum. Although oxidative stress and mitochondrial dysfunction have been implicated in the process, the mechanisms underlying the selective death of nigral dopaminergic neurons are still unknown.

Nuclear receptors constitute a conserved family of ligand activated transcription factors regulating gene expression. Several groups have provided numerous evidence suggesting an important involvement of a subgroup of nuclear receptors specifically associated with DA neurotransmission in the developing and mature CNS (for review, see Lévesque and Rouillard, 2007). This subgroup includes the Retinoid X Receptor (RXR) and orphan members of the thyroid/steroid nuclear receptor family named the Nur family, which includes Nurr1, Nur77, and Nor-1. Nurs are classified as early response genes and are induced by diverse signals, including growth factors, cytokines, peptide hormones, neurotransmitters and stress. Their ability to sense and rapidly respond to changes in the environment seems to be a hallmark of this subgroup (Maxwell and Muscat, 2006).

Nurr1 co-localizes with tyrosine hydroxylase (TH) and is essential for the development of midbrain DA neurons whereas Nur77 and Nor-1 expressions appear complementary to Nurr1 distribution, being constitutively expressed in the striatum (STR) and nucleus accumbens (NAC) (Lévesque and Rouillard, 2007). RXR is expressed in both SN and DA target structures (NAC and STR) and is essential as a common heterodimerization partner of several nuclear receptors including Nurr1 and Nur77 (Perlmann and Jansson, 1995; Maira et al., 1999). Indeed, Nurr1, as well as Nur77, can affect transcription of target genes by working alone as monomers or as homodimers, or by forming functional transcriptional complexes through heterodimerization with RXR partners (Perlmann and Jansson, 1995; Maira et al., 1999). Numerous evidence suggest that impaired Nurr1 function may be associated with an increased vulnerability of DA neurons to degeneration in PD (Le et al., 1999; Eells et al., 2002; Wallen-Mackenzie et al., 2003; Le et al., 2003; Jiang et al., 2005) whereas both Nur77 and Nor-1 are important signals for apoptosis pathways outside the CNS (Cheng et al., 1997). Nur77 was reported to play either an anti-apoptotic or pro-apoptotic function depending on the cellular context (Cheng et al., 1997; Li et al., 2000; Suzuki et al., 2003; Zhang, 2007). Indeed, Nur77 controls both survival and death of cancer cells (Zhang, 2007). A wealth of recent evidence demonstrates that Nur77 activities are regulated through its subcellular localization (Zhang, 2007). Outside the brain, Nur77 functions as a survival factor in the nucleus. In contrast, it is a potent killer when migrating to the mitochondria, where it binds to Bcl-2 and converts Bcl-2 from a protector to a killer, triggering release of cytochrome c and activating the caspase cascade (Li et al., 2000; Martinou et al., 2000; Lin et al., 2004; Zhang, 2007). Nur77-induced apoptosis is rapid and robust (Zhang, 2007). The contrasting actions of Nur77 on apoptosis are modulated by its intracellular localization which in turn is dependent upon the nature of the signal to which the cell is exposed (Zhang, 2007).

### SUMMARY OF THE INVENTION

The present invention therefore demonstrates that Nur77 is involved in neurotoxin-induced dopaminergic cell death via a mechanism involving its translocation from the nucleus to the cytoplasm. In vivo, Nur77 is rapidly and transiently induced after administration of the neurotoxin 6-hydroxydopamine (6-OHDA) in rats. The effects of MPTP are severely reduced in Nur77-deficient mice. In vitro, exposure of rat pheochromocytoma (PC12) cells to 6-OHDA or MPP+ induces Nur77 mRNA and protein expression and the translocation of Nur77 from the nucleus to the cytosol. Interestingly, 6-OHDA-induced cell death is significantly reduced by leptomycin B, a nuclear export inhibitor. We also report that docosahexaenoic acid (DHA), a RXR agonist, is able to prevent MPP+-induced PC12 cell death by blocking Nur77 nuclear export.

We hereby show that Nur77 can be induced in midbrain nigral dopamine neurons by administration of selective dopaminergic neurotoxins. Since Nur77 is modulated by a variety of signaling cascades including apoptotic and inflammatory signals, and its expression can be induced in the substantia nigra/ventral tegmental area (SN/VTA) complex. Also we have discovered that Nur77 is involved in the etiology and treatment of Parkinson's disease and that Nur77 and RXR, its heterodimerization partner can be targeted to protect nigral dopamine neurons from early cell death and therefore prevent neurodegenerative disorders such as Parkinson's disease.

It is therefore a first aspect, of the present invention to provide a method for identifying a compound for protecting/preventing dopaminergic cell-death, this method comprising the steps of assessing the ability of a compound to achieve at least one of: a) inhibit or prevent the expression of Nur77 in the nucleus of nigral dopamine neurons; b) inhibit or prevent the translocation of Nur77 to the cytosol of nigral dopamine neurons; c) increase or promote the transactivation of RXR in the nucleus of nigral dopaminergic neurons; or d) increase or promote the formation of RXR-Nur77 heterodimers in the nucleus of nigral dopaminergic neurons; whereby the ability of said compound to achieve at least one of said goal listed in a) to d) is an indication that said compound is a potential drug for preventing or inhibiting dopaminergic cell-death.

It is therefore a second aspect of the present invention to provide a method for protecting or preventing dopaminergic cell-death comprising the step of inhibiting or preventing Nur77-dependent apoptosis.

It is therefore a third aspect of the present invention to provide a method for the prevention of or protection from Parkinson's disease in a human comprising administering to the human a pharmaceutically effective amount of a compound or composition protecting or preventing Nur77-dependent apoptosis.

### DETAILED DESCRIPTION OF THE INVENTION

### Brief Description of the Drawings

Having thus generally described the aspects of the invention, reference will now be made to the accompanying drawings, showing by way of illustration, particular embodiments thereof, and in which:

FIG. 1 shows Nur77 expression in the substantia nigra (SN) after the administration of the neurotoxin 6-OHDA.

FIG. 2 shows Nur77 and Fluoro-Jade expression in the susbtantia nigra after 6-OHDA administration.

FIG. 3 shows the effects of MPTP in Nur77 (−/−) mice.

FIG. 4 shows Nur77 mRNA and 6-OHDA toxicity in PC12 cells.

FIG. 5 shows 6-OHDA-induced apoptosis in PC12 cells.

FIG. 6 shows Nur77 subcellular distribution after exposure to 6-OHDA.

FIG. 7 shows the effects of leptomycin B (LMB), a nuclear export inhibitor on Nur77 translocation and PC12 cell death.

FIG. 8 shows the translocation of Nur77 from the nucleus to the cytosol.

FIG. 9 schematically illustrates the pathway of Nur77 apoptosis and the effect of DHA on RXR nuclear transactivation in a nigral dopaminergic neuron.

### DEFINITIONS

The abbreviation “6-OHDA” means 6-hydroxydopamine.

The abbreviation “DHA” means docosahexaenoic acid.

The abbreviation “MPP⁺” means 1-methyl-4-phenylpyridinium ion. The abbreviation “MPTP” means 1-methyl-4-phenyl-1,2,3,6-tetrahydropyridine. The term Nur77 means a protein defined by sequence NM002135 in GENBANK®.

The terms “dopaminergic cells”; “nigral dopamine cells”, “dopamine neurons”, are all used interchangeably.

### DETAILED DESCRIPTION OF PARTICULAR EMBODIMENTS

### Method for Identifying Drugs for the Treatment of Parkinson's Disease

In this first aspect, there is provided a method for identifying a compound for protecting from, preventing or inhibiting dopaminergic cell-death, said method comprising the step of contacting a candidate compound with Nur-77 or a Nur-77-producing cell to evaluate the ability of said compound to bind to Nur77 or inhibit Nur77-dependent apoptosis wherein the ability of said compound to bind Nur77 or inhibit Nur77-dependent apoptosis is an indication that said compound is a potential drug for preventing or protecting from dopaminergic cell-death.

Particularly, the method comprises the step of contacting a candidate compound with Retinoid X Receptor (RXR) to evaluate the ability of the compound to bind to RXR (RXR-cpd), wherein the ability of the compound to bind to Nur77 is an indication that said compound is a potential drug for preventing/protecting dopaminergic cell-death.

More particularly, the method comprises the steps of: a) contacting a candidate compound with Retinoid X Receptor (RXR) to evaluate the ability of the compound to form a transcriptional complex with RXR (RXR-cpd); and b) assessing the ability of said transcriptional complex to inhibit the translocation of Nur77 (or Nur77-RXR heterodimer) to the cytosol of said dopaminergic cells; wherein the ability of the complex to inhibit Nur77 translocation is an indication that said compound is a potential drug for preventing/protecting dopaminergic cell-death.

Particularly, this method may be carried out in vitro using recombinant protein systems. Alternatively, this method may be carried out in situ using cells that express RXR and Nur77. These cells may express these proteins constitutively or transiently. Particularly, these cells are dopaminergic neurons in culture or PC12 cells. More particularly, these cells are PC12 cells.

### Method of Prevention

The present invention therefore provides a method for protecting/preventing dopaminergic cell-death comprising the step of inhibiting Nur77-dependent apoptosis. Particularly, the invention provides a method for protecting/preventing dopaminergic cell-death comprising the step of inhibiting the expression of Nur77 in the nucleus of nigral dopamine neurons. More particularly, there is provided a method for protecting/preventing dopaminergic cell-death comprising the step of inhibiting the translocation of Nur77 to the cytosol of nigral dopamine neurons. Still, particularly, the invention provides a method for protecting/preventing dopaminergic cell-death comprising the step of increasing the transactivation of RXR in the nucleus of nigral dopaminergic neurons. Alternatively, there is provided a method for protecting/preventing dopaminergic cell-death comprising the step of increasing or promoting the formation of RXR-Nur77 heterodimers in the nucleus of nigral dopaminergic neurons.

### Method of Treatment

In a third aspect, the invention provides a method for the prevention of Parkinson's disease in a subject comprising administering to the subject a pharmaceutically effective amount of a compound inhibiting Nur77-dependent apoptosis. Particularly, the compound inhibiting Nur-77 dependent apoptosis is a Retinoid X Receptor ligand. More particularly, the RXR ligand induces the transactivation of the RXR in the nucleus of nigral dopamine neurons favoring the formation of a RXR-Nur77 complex. More particularly, the RXR ligand blocks the translocation of the RXR-Nur77 complex to the cytosol of nigral dopamine neurons.

### Subject

Particularly, the subject is a human.

### Parkinson's Disease Symptoms

Particularly, the treatment may be administered before or after symptoms appear in the human. Particularly, the symptoms of Parkinson's disease can be one or more than one of the following: slowness of movement, compromise of balance, muscle rigidity and tremor.

### Compounds for the Treatment or Prevention of Parkinson's Disease

Particularly, the compound administered is an RXR ligand. More particularly, the RXR ligand is docosahexaenoic acid (DHA) or its metabolites or derivatives such as, for example, DEA (docosaenoic acid).

Parkinson's disease is a progressive neurodegenerative disorder characterized by a selective death of nigral dopamine neurons. Currently, the mechanisms underlying the neurodegenerative process remain unclear. Nur77 is a transcription factor closely associated to dopamine neurotransmission in the mature brain. However, Nur77 is also a potent signal for apoptosis outside the CNS. This nuclear receptor is a survival factor when located in the nucleus whereas it is a potent killer when migrating to the mitochondria. Applicant reports that Nur77 is directly involved in dopamine cell death induced by specific neurotoxins. Indeed, Nur77 is rapidly and transiently induced in midbrain dopamine neurons after the administration of 6-OHDA in vivo. In addition, in vitro data demonstrate that Nur77 is involved in cell death after 6-OHDA and MPTP exposure via a mechanism involving its translocation from the nucleus to the cytosol. 6-OHDA- and MPTP-induced cell death in vitro is then significantly reduced by administration of docosahexaenoic acid (DHA), a polyunsaturated fatty acid binding the Retinoid X Receptor (RXR). This nuclear receptor interacts with Nur77 by forming transcriptional complexes through heterodimerization. Applicant also demonstrates that transactivation of RXR by DHA silences the translocation of Nur77 to the cytosol thereby inhibiting Nur77-dependent apoptosis. Altogether, these data suggest that Nur77 is involved in dopamine cell death via a mechanism that can be blocked by the administration of DHA. These novel findings open the way to new therapeutic strategies that may slow or stop the progression of Parkinson's disease.

The present invention to provide a method for protecting/preventing dopaminergic cell-death comprising the step of inhibiting the translocation of Nur77 in the cytosol. This can be achieved by a) increasing the transactivation of RXR in the nucleus of nigral dopaminergic neurons; b) increasing or promoting the formation of RXR-Nur77 heterodimers in the nucleus of nigral dopaminergic neurons; or c) inhibiting the translocation of Nur77 from the nucleus to the cytosol. Either one, or all of these mechanism may be exploited in the search for a therapeutic agent effective against Parkinson's disease.

### EXAMPLES

### Materials & Methods

6-hydroxydopamine Lesions in Rats

Fifteen male Sprague—Dawley albino rats (Charles River, Montreal, Canada) weighing 250-275 g at the beginning of the experiment were used in this study. They were housed two per cage in a temperature-controlled environment maintained under a 12-h light/dark cycle with ad libitum access to food and water. Handling of the rats was performed in accordance with the Canadian Guide for the Care and Use of Laboratory animals and all procedures were approved by the Institutional Animal Care Committee of Laval University. All efforts were made to minimize the number of animals used and their suffering. Rats were anaesthetized in an induction chamber with 4% isoflurane in 95% O₂/5% CO₂ before being transferred to a stereotaxic frame and maintained at 1.5-3% isoflurane. A sagittal cut exposed bregma and a hole was drilled. 6-OHDA was injected into the right striatum (AP: 0.6 with respect to bregma, L: 3.0 and V: 4.5) or into the right median forebrain bundle (AP: −4.3; ML: 1.4; and DV: 8.0 (Paxinos and Watson, 1986)). The neurotoxin 6-OHDA (12 μg/3 μl) was dissolved in saline containing ascorbic acid (0.1%) and injected at a rate of 1 μl/min and the syringe was left in place for an additional 5 min to allow diffusion of 6-OHDA away from the needle tip. Rats were anaesthetized with CO₂ and killed by decapitation at 3, 9 24, 72 and 120 hrs after 6-OHDA administration.

### In Situ Hybridization

Cryostat-cut brain sections (12 μm) were mounted on Superfrost slides (Fisher Scientific, Toronto, ON, Canada) and stored at −80° C. Specific [³⁵S]UTP-labeled complementary.

RNA (cRNA) probes were used and preparation of the Nur77 probe was performed as described previously (Beaudry et al., 2000). The antisense cRNA probe for Nur77 stems from a 2.4-kb EcoRI fragment of a full-length rat Nur77 cDNA subcloned into pBluescript SK-1 and linearized with BamHI. The single-stranded riboprobe complementary to the RNA of interest was synthesized and labeled using Promega riboprobe kit (Promega, Madison, Wis.), [³⁵S]UTP (Perkin Elmer Inc., Canada), and the RNA polymerase T3. The hybridization technique was performed as previously described (Beaudry et al., 2000; Langlois et al., 2001). Briefly, brain sections were fixed in a 20-min paraformaldehyde bath (4% paraformaldehyde in 100 mM phosphate buffer) and then rinsed twice in phosphate buffer (100 mM) for 5 min. Subsequently, a triethanolamine incubation (TEA 100 mM, pH 8.0) and a 10-min acetylation bath (0.25% acetic anhydride, TEA 100 mM) followed. Slides were finally rinsed in standard saline citrate (SSC) (300 mM NaCl, 30 mM sodium citrate) and dehydrated in increasing concentrations of ethanol. The ³⁵S-labeled riboprobe was added to a concentration of 2×10⁷ cpm/ml in a hybridization mix (Denhart's solution, dextran sulfate, and 50% deionized formamide) and heated at 65° C. for 5 min. Each slide was covered up with 50 μl of the hybridization solution and coverslipped. The hybridization took place overnight at 58° C. on a slide warmer. After hybridization, slides were soaked in SSC (600 mM NaCl, 30 mM sodium citrate) for 30 min to remove coverslips and then washed in four subsequent SSC baths. Afterward, slides spent 30 min in a 37° C. incubation buffer with RNAse A (2 mg/100 ml). A series of SSC baths with dithiothreitol (1 mM) followed, one of which was at 55° C. for 30 min. Finally, tissues were dehydrated in ethanol, dried up, and apposed to a Biomax MR film (Kodak). Autoradiograms were developed approximately 48 h later. Subsequently, sections were dipped in NTB2 nuclear emulsion (diluted 1:1 with distilled water; Kodak). Slides air-dried for 4 h and stored in the dark for 5 days at 4° C. The emulsion was then developed in D19 developer (Kodak) for 3.5 min at 14-15° C. and fixed in Rapid Fixer (Kodak) for 5 min. Thereafter, brain tissue was rinsed in running distilled water for 1-2 h and dehydrated trough graded concentration of alcohol, cleared in xylene and coverslipped with DPX.

### Fluoro-Jade Coloration and DAPI

The brain sections were mounted on gelatin-coated slides (Brain Research Laboratories, Newton, Mass.) and air-dried overnight. They were dehydrated in ascending grades of ethanol, hydrated in descending grades of ethanol and finally rinsed in milliQ water. Afterwards, the sections were dipped in a 0.06% potassium permanganate solution bath for 10 min and washed in milliQ water. They were then incubated in a milliQ solution containing 0.0002% (v/v) DAPI (Molecular Probes, Eugene, Oreg.), 0.1% (v/v) acetic acid glacial and 0.0004% (v/v) Fluoro-Jade B (Chemicon, Temecula, Calif.) for 20 min (Schmued et al., 1997). After a few rinses in milliQ water, they were completely air-dried, dipped in xylene and coverslipped with DPX. Degenerating neurons and fibers appeared green and nuclei emerged blue in fluorescence.

### MPTP Lesions in Mice

Wild-type (Nur77(+/+)) male (C57BL/6, weighing 20-25 g) mice were purchased from Charles River, Canada. Nur77 knockout (Nur77(−/−)) mice were developed and graciously provided by Dr Jeff Milbrandt at the Washington University, St. Louis, Mo., USA (Lee et al., 1995). These mice are healthy and reproduce normally (Crawford et al., 1995; Lee et al., 1995). They were produced in a mixed background and have been backcrossed into the C57BL/6 strain for at least ten generations to reduce genetic background heterogeneity (Jeff Milbrandt, personal communication). We maintain a Nur77(−/−) mouse colony at the animal care facility of our research center. Young adult Nur77(−/−) male mice weighing 20-25 g were used for present experiments. All mice were housed three or four per cage in a temperature-controlled environment and maintained under a 12-h light-dark cycle with ad libitum access to food and water.

In total, 17 mice (8 Nur77 (+/+) and 9 Nur77 (−/−)) received seven intraperitoneal (i.p.) injections of MPTP-HCl (20 mg/kg free base; Sigma, St. Louis, Mo.) dissolved in NaCl 0.9%. MPTP was administered twice on the first 2 days of the experimental protocol at an interval of 12 h and once a day on 3 subsequent days (Tremblay et al. 2006; Gibrat et al., 2010). Animals were ultimately sacrificed 14 days after the last injection. An identical number of mice (8 Nur77 (+/+) and 9 Nur77 (−/−)) received i.p. saline injections. All animals were sacrificed 14 days after the last injection.

### Post Mortem Histological Evaluation

Animals were sacrificed under deep anesthesia with sodium pentobarbital [60 mg/ml, i.p. (0.1 ml/100 g)] and perfused via intracardiac infusion with heparinized saline (0.9%) followed by 4% paraformaldehyde (PFA) with Borax (sodium tetraborate) in milliQ water, pH 9.5, 14 days after the last MPTP injection. After intracardiac perfusion, brains were collected and post-fixed in 4% PFA for 6 h and transferred to 20% sucrose in 0.1M phosphate buffer saline (PBS) for cryoprotection. Coronal brain sections of 30 m thickness were cut on a freezing microtome (Leica Microsystems, Montreal, Que.), serially collected in anti-freeze solution (monophosphate sodium monobasic 0.2 M, monophosphate sodium dibasic 0.2 M, ethylen glycol 30%, glycerol 20%). At the beginning of each immunostaining protocol, sections were washed in PBS 0.1M and then placed in 3% peroxide for 30 min at room temperature (RT). Slices were transferred in 0.1M PBS for several washes and then preincubated in a 0.1M PBS solution containing 0.1% Triton X-100 (Sigma) and 5% normal goat serum or normal donkey serum (NGS or NDS) for 30 min at RT. Sections were incubated overnight at 4.0 with a rabbit anti-TH antibody (Pel-Freez, Rogers, AR; 1:5000). After overnight incubation at 4° C. with the primary antibody, sections were washed in PBS 0.1M and incubated for 1 h at RT in a PBS solution containing 0.1% Triton X-100, 5% NGS or NDS, and biotinylated goat anti-rabbit IgG (Vector Laboratories, Burlington, ON; 1:1500) for thyronine hydrolxylase (TH). Following three washes in PBS, sections were placed in a solution containing avidin-biotin peroxidase complex (ABC Elite kit; Vector Laboratories, Burlington, ON) for 1 h at RT. The bound antibodies were visualized by placing the sections in a Tris 0.05M buffer solution containing 0.5 mg/ml of 3,3′ diaminobenzidine tetrahydrochloride (DAB; Sigma) and 0.1% of 30% hydrogen peroxide (Sigma) at room temperature. The reaction was stopped by washing in 0.05M Tris buffer and subsequent PBS washes. Sections were then mounted on gelatin-coated slides and counterstained with cresyl violet (Sigma). Finally, all sections were ultimately air-dried, dehydrated in ascending grades of ethanol, cleaned in xylene and coverslipped with DPX mounting media (Electron Microscopy Science, Hatfield, Pa.).

### Stereological Counts of Dopamine Neurons

The loss of DA neurons was determined by counting TH-immunoreactive cells under bright-field illumination. After delineation of the SNpc at low magnification (10×), three sections from the entire region were sampled at higher magnification (20×) using Stereo investigator software (Microbrightfield, Colchester, Vt.) attached to a E800 Nikon microscope (Nikon Instruments, Toronto, Canada). Section sampling was performed based on the achievement of the desired coefficient of error (Glaser and Glaser, 2000), which ensured the correct representations of cell counts on three sections of the same brain (AP levels: −5.3 to −6.04 mm) (Paxinos and Watson, 1986). The optical fractionator method (Glaser and Glaser, 2000) was used to count TH-positive (TH+ and cresyl violet+neurons) and TH-negative (cresyl violet+only) expressing cells. The entire depth of the field was sampled ignoring the upper and lower 2 m to avoid counting cells that might be missing nuclei. Cell counts were performed blindly by two independent investigators. Note that these analyses were restricted to the SNpc and thus excluded the ventral tegmental area (VTA). The average density of TH-immunoreactive expressing neurons per section, derived from the analysis of three SNpc sections, was ultimately plotted.

### Materials for In Vitro Experiments

Fetal calf and horse serums were obtained from Life Technologies (Ontario, Canada). Culture medium RPMI 1640, MPP+, gentamicin, and GenElute Total mammalian RNA extraction kit were purchased from Sigma Chemicals (Ontario, Canada). RT reagents were purchased from New England Biolab (NE, USA). PCR reagent HotMasterMix was purchased from Eppendorf (Westbury, N.Y., USA). Polyclonal rabbit anti-Nur77 antibody (epitope corresponding to amino acids 59-269 of mouse Nur77) was purchased at Santa Cruz Biotechnology (Santa Cruz, Calif., USA).

Monoclonal mouse anti-TH antibody (clone 2) was purchased from Sigma Chemicals (Ontario, Canada). Polyclonal rabbit anti-HDAC1 antibody (epitope corresponding to amino acids 450 to C-terminus of human histone deacetylase 1 (HDAC1) was purchased from Abcam (Cambridge, Mass., USA). Anti-Rabbit Cy3-conjugated secondary antibody was purchased from Jackson ImmunoResearch (West Grove, Pa., USA) and Prolong® Antifade from Molecular Probes (Eugene, Oreg., USA).

### Cell Culture and Treatments

PC12 cells were obtained from ATCC CRL 1721 (VA, USA) and were grown in phenol red-free RPMI 1640 medium supplemented with 5% fetal calf serum (FCS), 10% horse serum and gentamicin (50 μg/ml). They were cultured on Primaria 6-well plates (BD Falcon, Ontario, Canada) for RT-PCR experiments and immunoblotting; on Primaria 96-wells plates for LDH cytoxicity experiments and finally on poly-L-lysine-coated circular glass coverslips for immunocytofluorescence assays. Cells were fed fresh medium every 3 days and maintained at 37° C. in a humidified 5% CO₂ atmosphere. Treatment medium included RPMI 1640, 1% FCS (charcoal stripped to remove steroids), gentamicin, 6-OHDA (10-30 μM). Control conditions were without toxin. The 6-OHDA concentration used (30 μM) was chosen since it did not induce more than 70% cell death after 24 h, as revealed by dose-response and time course studies (data not shown). For the present study, kinetic time periods were: 0 h, 3 h, 6 h, 9 h, 12 h and 24 h. After each experiment, culture medium was harvested for cytotoxicity detection and cells were collected for RT-PCR and ICF/Fractioning (Gelinas & Martinoli 2002; Gagne et al. 2003; Gelinas et al. 2004; Chiasson et al. 2006).

### Cytotoxicity and Metabolic Activity

Cytotoxicity was evaluated by colorimetric assay based on the measurement of lactate deshydrogenase (LDH) activity, as previously described (Chiasson et al. 2006). Briefly, media were collected and centrifuged at 800×g at 4° C. for 2 min. The cell-free supernatant (100 μl) was then used to determine LDH activity by measuring the absorbance at a wavelength of 490 nm (A490) on a microplate reader (Multiscan Ascent Microplate reader, Thermolab System, Franklin Mass.) according to the working procedures of Roche's protocol. Total cellular LDH was determined by lysing cells with 1% Triton X-100. The A490 is proportional to the number of viable cells in the culture (Roehm et al. 1991).

### RT-PCR

Total RNA was extracted with Sigma's GenElute Mammalian Total RNA extraction kit. RNA was assayed for each condition, and reverse transcriptase (RT) was performed with 1 μg total RNA. PCR was then performed with 10% of the RT product, amplified with specific primers and HotMasterMix. For specific semiquantitative RT-PCR of transcripts, forward and reverse primers were synthesized at Sigma Genosys (The Woodlands, Tex., US) and are shown in Table 1.

TABLE 1

| | | | SEQ | | |
|---|---|---|---|---|---|
| | Expected | | ID | Annealing | |
| Name | MW | Sequences | NO. | temp | References |
| Nur77 | 696 bp | 5′-AGG ACC GCG TGA CCA CCT-3′ | 1 | 52° C. | (Beaudry et |
| | | 5′-CGT CCA GAA TGC CGG A-3′ | 2 | | al. 2000) |
| Bcl-2 | 270 bp | 5′-CGACTTTGCAGAGATGTCCA-3′ | 3 | 60° C. | (Ando et al. |
| | | 5′-CATCCACAGAGCGATGTTGT-3′ | 4 | | 2005) |
| Bax | 208 bp | 5′-TGC AGA GGA TGA TTG CTG AC-3′ | 5 | 56° C. | (Ando et al. |
| | | 5′-GGA GGA AGT CCA GTG TCC AG-3′ | 6 | | 2005) |
| p53 | 177 bp | 5′-GTC TAC GTC CCG CCA TAA AA-3′ | 7 | 54° C. | (Ando et al. |
| | | 5′-AGG CAG TGA AGG GAC TAG CA-3′ | 8 | | 2005) |
| 18S | 150 bp | 5′-GTA ACC CGT TGA ACC CCA TT-3′ | 9 | 55° C. | (Schmittgen & |
| | | 5′-CCA TCC AAT CGG TAG TAG CG-3′ | 10 | | Zakrajsek |
| | | | | | 2000) |

The standardized amplification protocol consisted of an initial denaturation step at 94° C. for 2 min, followed by 25 sequential cycles of 94° C. for 1 min, specific annealing temperature (Table 1) for 1 min and 65° C. for 1.5 min, with a final extension of 7 min at 65° C. Amplification was carried out in a Perkin Elmer GeneAmp PCR System 2400 thermal cycler. To ensure that quantification was achieved in an exponential phase, a range of target and standard amounts were amplified over a range of cycles. The final number of cycles was chosen as the time in which the amplified products were still accumulating in a logarithmic fashion, i.e. the system was in the exponential phase, as already described (Rousseau et al. 2002). 10 μl of the PCR products were analyzed by electrophoresis on 1.5% agarose gel in 1×TAE running buffer (40 mM Tris-acetate, 1 mM EDTA, pH 8.0) and stained with ethidium bromide. Images were acquired with a Fluor SP Imager from Alpha Innotech, and analyzed with their Fluor Chem SP software. Every analysis was correlated with the amplicon of ribosomal 18S subunit as internal amplification controls.

### Immunocytofluorescence and TUNEL

PC12 cells were grown and treated on circular glass coverslips, then fixed in 4% paraformaldehyde for 1 hour at 4° C. Immunofluorescence detection sequence was as follows: primary antibodies (1:1000, 1 h at RT), then slides were washed with PBS and stained with a fluorescent dyed secondary antibody (Cy3, 1:50) for 2 h at RT. 4′,6-Diamidino-2-phenylindole (DAPI) was used to counterstain all nuclei. They were washed 3 times with PBS. Finally, slides were mounted with the Prolong® Antifade Kit (Molecular Probes, Eugene, Oreg.). Images were acquired with an Olympus IX-70 inverted microscope with a high-pressure mercury burner and necessary filter cubes (Olympus U-MNUV, U-MNIBA and U-MWIG), and were analyzed with Bio-Rad's QuantityOne software. TUNEL assay was performed on the same slides as with the immunofluorescence (Heatwole 1999).

### Nuclei-Cytoplasmic Fractioning

PC12 cells were grown and treated in 6-wells plates. We used a nuclear extraction kit (Active Motif, Carlsbad, Calif.) to separate cytoplasmic protein content from nuclear protein content. Briefly, cells were washed in wells with a mixture of PBS and phosphatase inhibitors, and then harvested in centrifuge tubes. Hypotonic buffer swelled cytoplasmic membrane, which an afterward mild detergent ruptured.

Centrifugation at 14,000×g pelleted the intact nuclei, and soluble material was preserved as the cytoplasmic fraction. Nuclei were the lysed with the provided lysis buffer. Protein dosage was performed with a bicinchoninic acid (BCA)-based SDS-compatible Protein Assay Kit (Pierce, Rockfort, Ill.) on each fraction of each sample.

### Electrophoresis and Immunoblot Analysis

Equal amounts of nuclei or cytoplasmic protein fractions were loaded onto a 10% polyacrylamide-SDS gel. After electrophoretic separation (180 volts, 45 min.) the polyacrylamide gels were transferred onto nylon (PVDF) membranes (0.22 μm pore size, Bio-Rad) at 50 V for 2 h. The membranes were blocked with 5% non-fat powder milk for 1 h. Immunoblotting was probed with primary antibody for at least 2 h. Dilution of anti-Nur77, anti-TH and Anti-HDAC1 was 1:1000, 1:1000 and 1:1250, respectively. Horseradish peroxydase (POD)-conjugated secondary antibody was added for 2 h. Immunopositive signal was visualized with an enhanced chemiluminescence method (Roche, Que., Canada) and were acquired with a Fluor SP Imager from Alpha Innotech. Quantitative analysis was performed with the Fluor Chem SP software.

### Data Analysis and Statistics

The statistical significance was assessed against control using unpaired Student test with a two-tailed P value using GraphPad InStat version 3.06 for Windows© (San Diego Calif. USA, www.graphpad.com). Data are expressed as mean±S.E.M from 3 to 10 independent experiments. Stars (*) are for statistical difference between treatment and respective control condition.

### Results

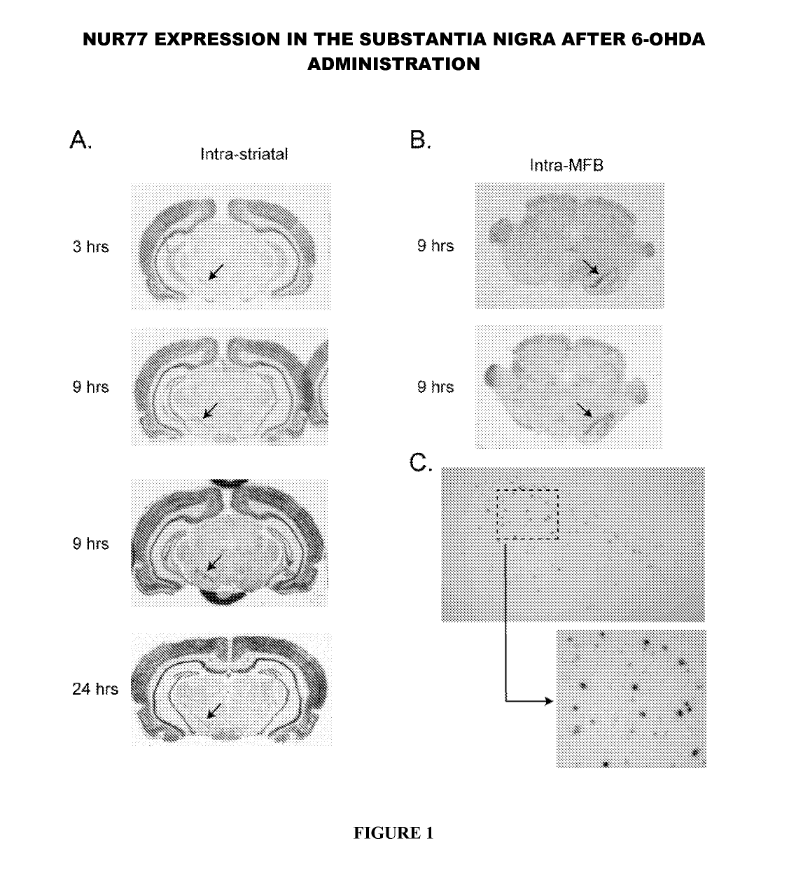

In FIG. 1 (A), we can see the black arrow indicating the SN on the lesioned side. (B) Nur77 mRNA expression in the SN after 6-OHDA administration into the medial forebrain bundle (Intra-MFB). (C) Nur77 mRNA expression in the SN in a 6-OHDA rat 9 hrs after intra-striatal administration of the neurotoxin. Note the moderate number of Nur77-positive cells. However, Nur77 is highly expressed in these Nur77-positive cells.

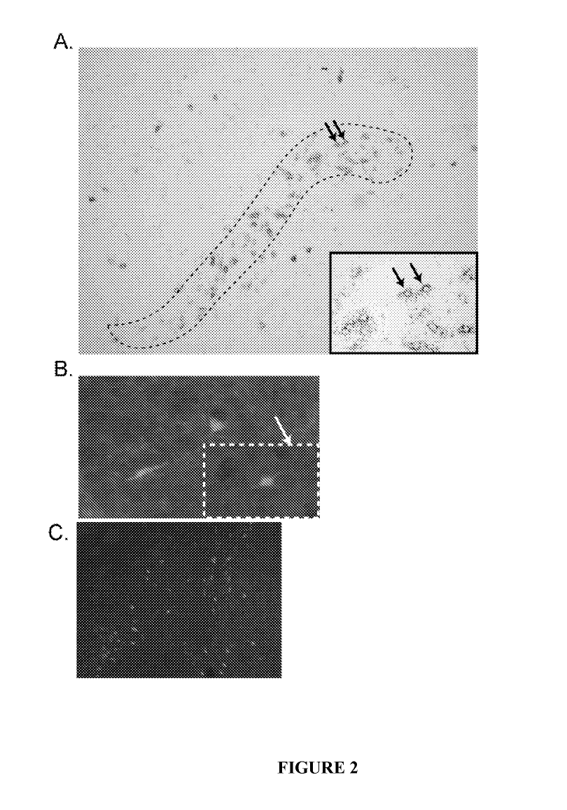

In FIG. 2(A), we show a brightfield image of Nur77 mRNA in the substantia nigra of a 6-OHDA rat sacrificed 9 hrs after the neurotoxin administration. The image was taken from a brain slice dipped into NTB-2 emulsion milk (Kodak). Black arrows indicate a pair of neurons shown at higher magnification in the inset. Note that the nucleus seems to be devoid of autoradiographic grains. Interestingly, to induce apoptosis outside the brain, Nur77 is translocated from the nucleus to the mitochondria. B: Fluoro-Jade B labelling of degenerating cells in the substantia nigra in the same rat sacrificed 9 hours after 6-OHDA administration. White arrows indicate Fluoro-Jade B-positive cells. The inset shows another Fluoro-Jade B positive cell that was located more medially in the substantia nigra. C. Fluoro-Jade B labelling in another rat sacrificed 48 h after 6-OHDA. Fluorescein isothiocyanate (FITC) filter.

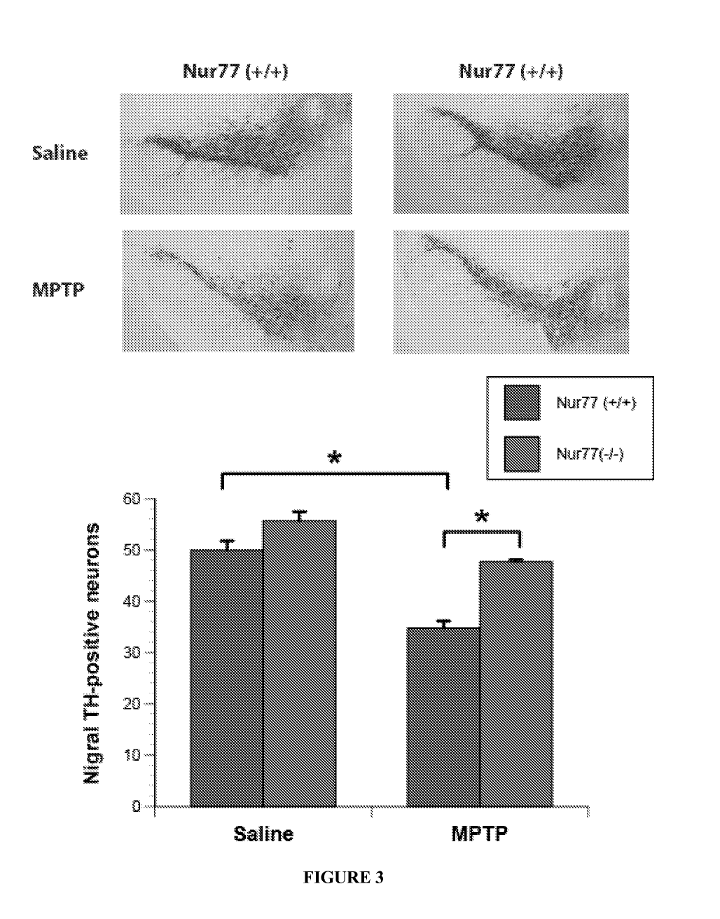

In FIG. 3, 17 mice (8 Nur77 (+/+) and 9 Nur77 (−/−)) received a total of 7 i.p. injections of MPTP-HCl (20 mg/kg free base) dissolved in saline. MPTP was administered twice on the first 2 days and once on the three subsequent days. An identical number of mice (8 Nur77 (+/+) and 9 Nur77 (−/−)) received i.p. saline injections. All animals were sacrificed 14 days after the last injection. Animals were perfused and coronal 30 μm sections were cut on a freezing microtome. Tyrosine hydroxylase (TH) immunohistochemistry and stereology was performed as described (Tremblay et al., 2006; Gibrat et al. 2010). The loss of DA neurons was determined by counting TH-immunoreactive cells under bright-field illumination using the Stereo investigator software attached to a Nikon E800 microscope. Statistical analysis (2-way ANOVA) revealed significant effects for the factors mouse (F(1,34)=4.20 P=0.048) and MPTP treatment (F(1,34)=6.58 P=0.015) without interactions between these two factors (F(1,34)=0.626 P=0.424). Post-hoc analysis indicate that MPTP significantly reduced the number of nigral TH neurons (P<0.05) in Nur77 (+/+) mice but not in Nur77 (−/−) mice. There was a significant difference in the number of TH-positive cells in Nur77 (+/+) mice treated with MPTP compared to Nur77 (−/−) MPTP-treated mice. * P<0.05

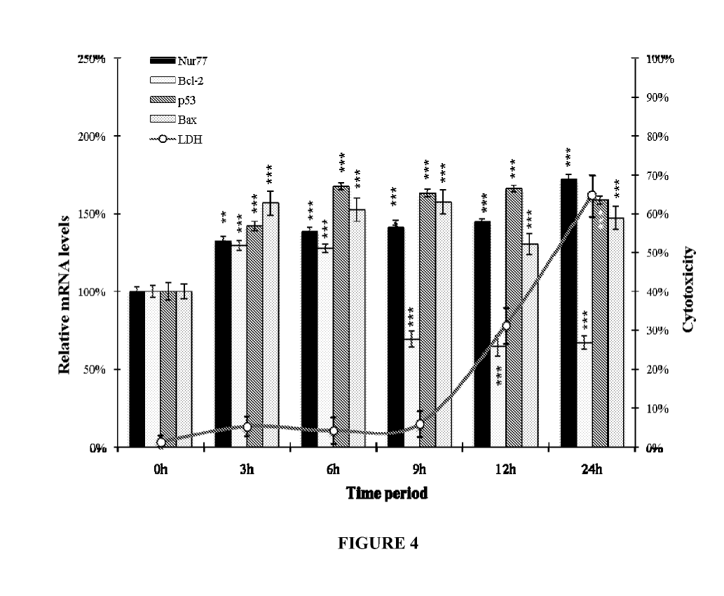

In FIG. 4, the histogram shows the mRNA expression of Nur77, Bcl-2, p53 and BAX after treatment of PC12 cells with 10 μM 6-OHDA. The solid line depicts the concurrent cytotoxicity kinetic as measured by LDH activity. There is a significant rise of Nur77, Bcl-2, p53 and BAx expression 3 h after the exposure to the neurotoxin. Levels of Nur77, p53 and BAx remain elevated for up to 24 hrs whereas the expression of Bcl-2, an anti-apoptotic marker, decreases to 50% of the control level at 9 hrs and remain downregulated at 12 and 24 h. Cytotoxicity measurements demonstrate an expotential and significant rise of cellular death starting at 12 h after the exposure to 6-OHDA.

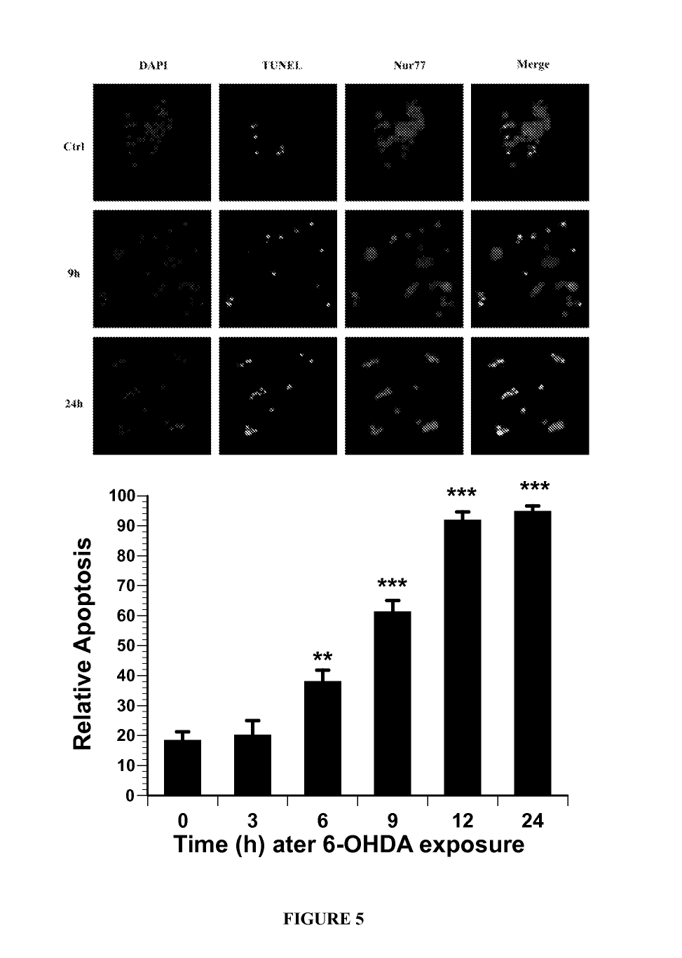

In FIG. 5, we can quantify apoptosis with the expression of TUNEL/DAPI. In vitro exposure of PC12 cells to the neurotoxin 6-OHDA induces a significant increase of apoptosis after 6, 12 and 24 h. At 12 and 24 h, more than 90% of the cells show signs of apoptosis (co-localization of DAPI and TUNEL). Nur77 is co-expressed with TUNEL-DAPI in a very high percentage of these cells.

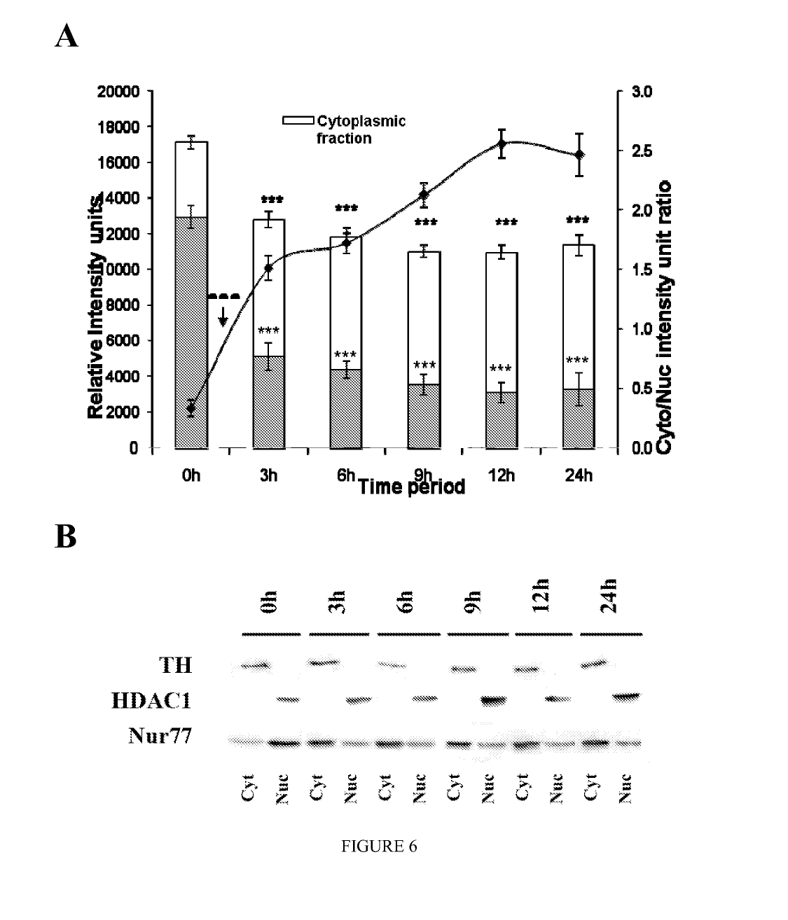

In FIG. 6, we can observe that, upon 6-OHDA treatment, there is a rapid translocation of Nur77 from the nucleus to the cytosol. At time 0, a larger fraction of Nur77 protein (Western Blot; panel B) is located into the nuclear fraction (grey bars in panel A; ratio cytosol/nucleus=0.33±0.07). Beginning at 3 h after 6-OHDA exposure, the cytosolic fraction signal (white bars) rises significantly and the ratio cytosol/nucleus is reversed (ratio at 3 h: 1.51±0.1, 6 h: 1.72±0.09, 9 h: 2.13±0.1, 12 h: 2.56±0.12 and 24 h: 2.47±18). This clearly indicates that exposure of PC12 cells to 6-OHDA induces a rapid translocation of Nur77 from the nucleus to the cytosol.

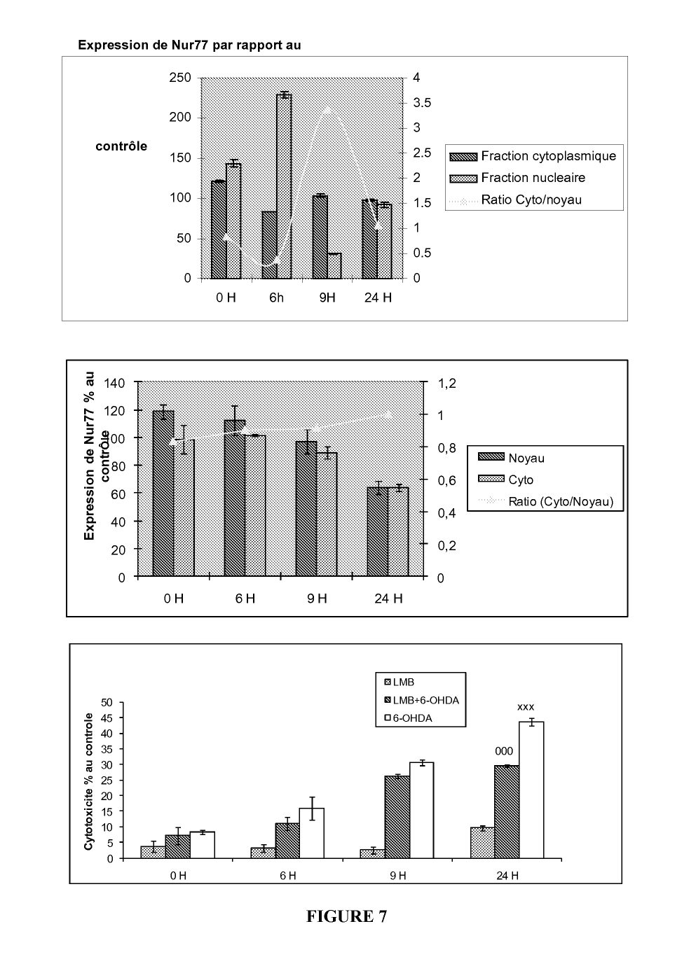

In FIG. 7(A), as previously demonstrated, incubation of PC12 cells with 6-OHDA (30 μM) induced an upregulation of Nu77 and its translocation from the nucleus to the cystosol starting at 9 h. (B) This translocation is blocked by co-incubation with LMB (1 nM). (C) Co-incubation of LMB with 6-OHDA induced a significant reduction of 6-OHDA induced cell death at 24 h. LMB alone has no effect on PC12 cell survival.

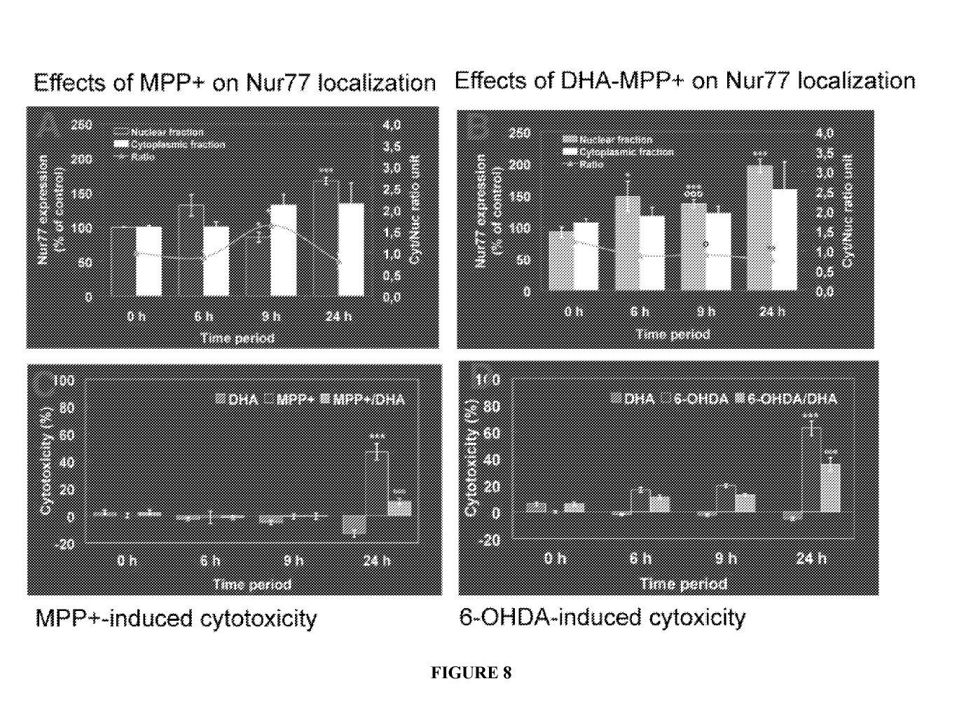

In FIG. 8(A), we observe that, upon exposure to MPP+(5 mM), there is a rapid translocation of Nur77 protein from the nucleus to the cytosol beginning at 6 hrs after MPP+. The cytosilic fraction signal (white bars) rises significantly and the ratio cytosol/nucleus is reversed. (B) Translocation of Nur77 from the nucleus to the cytosol is blocked by co-incubation of MPP+ with DHA (10⁻⁶ M). (C and D) The cytotoxicity induced by MPP++ (5 mM) or 6-OHDA (30 μM) in PC12cells is significantly reduced by co-incubation with DHA (10⁻⁶ M).

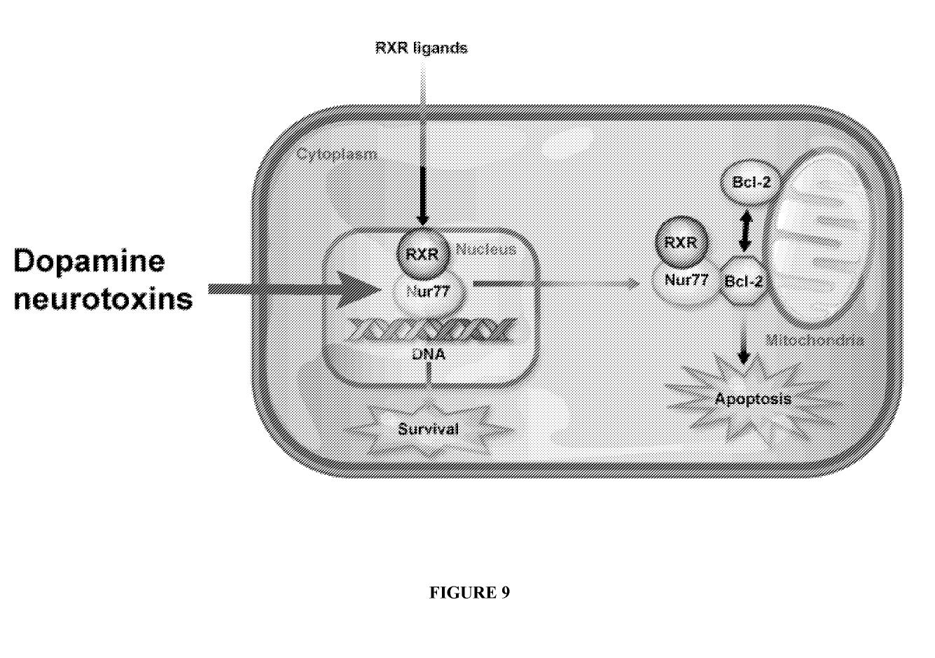

As represented in FIG. 9, Nur77-in dopamine cell death and neuroprotection. Nur77 is induced in nigral dopamine neurons by dopamine neurotoxins. Subsequently, Nur77 is translocated from the nucleus to the mitochondria where it interacts with Bcl-2, triggers cytochrome C release and activates of the caspases cascade. Activation of RXR-Nur77 heterodimers by RXR agonists silences the nuclear export signal thus causing nuclear localization, inhibition of mitochondrial targeting and suppression of apoptosis. Alternatively, RXR agonists might inhibit apoptosis due to the inability of RXR-Nur77 heterodimers to interact with the mitochondria.

### Discussion

The origin of cell degeneration in Parkinson's disease is largely unknown. This study provides several lines of evidence in vivo and in vitro that Nur77 is involved in dopaminergic cell death via its upregulation and a mechanism involving its translocation from the nucleus to the cytosol. We also provide evidence that Nur77-dependent apoptosis can be significantly reduced by silencing its translocation to the cytosol.

Numerous data suggest that impaired Nurr1 function may be associated with an increased vulnerability of dopamine neurons to degeneration in PD (Le et al., 1999; Le et al., 2003; Eells et al., 2002; Jiang et al., 2005) and Nurr1 is required for maintenance of maturing and adult midbrain dopamine neurons (Kadkhodaei et al., 2009). However, little is known about the role of Nur77 despite the fact that it is an important signal for apoptosis pathways outside the brain (Cheng et al., 1997). There is a possibility that Nur77 might be involved in environmental-induced dopamine-neuronal death since this nuclear receptor/transcription factor is modulated by a variety of signaling cascades (Fass et al., 2003; Bourhis et al., 2008; Kovalovsky et al., 2002; Darragh et al., 2005) and its expression can be induced in the substantia nigra/ventral tegmental area complex (Maheux et al., 2005; Bourhis et al., 2008). Although, Nur77 is not normally expressed in the substantia nigra/ventral tegmental area complex, we demonstrate here that Nur77 is rapidly and transiently induced in midbrain dopaminergic neurons after intracerebral 6-OHDA administration in rats. Nur77 mRNA is induced as early as 3 h after 6-OHDA administration. Its regulation in this brain area by 6-OHDA is in accordance with recent data demonstrating that Nur77 can be induced in the SN/VTA complex by antipsychotic drugs (Maheux et al., 2005; Bourhis et al., 2008). Nur77 induction in midbrain dopaminergic neurons by the neurotoxin 6-OHDA raises the possibility of its involvement in dopaminergic cell death. Interestingly, Fluoro-Jade staining, a marker of neuronal death (Schmued and Hopkins, 2000), is also observed at 3 h after intracerebral 6-OHDA administration and is maximal at 48 h suggesting similar time courses for Nur77 expression and signs of apoptosis. A direct involvement of Nur77 in neurotoxin-induced neuronal death is also supported by the fact that the neurotoxic effects of a MPTP systemic regime are severely reduced in Nur77 (−/−) mice.

Cytotoxicity in undiffererentiated and differentiated PC12 cells is a suitable in vitro model of Parkinson's disease (Hirata et al., 2006; Yamamoto et al., 2007). PC12 cells synthesize, store, release and metabolize dopamine. We show here that Nur77 mRNA is rapidly and significantly upregulated in PC12 cells after exposure to 6-OHDA. This upregulation of Nur77 begins as early as 3 h following exposure to the neurotoxin and precedes the signs of apoptosis which are significantly elevated starting at 6 h. The upregulation of Nur77 is accompanied by significant changes in the levels of other apoptotic genes. Levels of Nur77, p53 and Bax rose at 3 h after exposure to the neurotoxin and remained elevated up to 24 h whereas the expression of Bcl-2, an anti-apoptotic marker, decreased to 50% of the control level at 9 h and remained down-regulated at 12 and 24 h. At this time period, more than 90% of the cells show signs of apoptosis (TUNEL/DAPI) and Nur77 is co-expressed with these apoptosis markers in a very high percentage of these cells.

As recently reported in SH-SY5Y cells (No et al., 2010), we report that upon the treatment of PC12 cells with 6-OHDA, there is a rapid translocation of the Nur77 protein from the nucleus to the cytosol. The drastic change in the subcellular distribution of Nur77 correlates well with the time-course of 6-OHDA-induced neurotoxicity. Interestingly, 6-OHDA-induced Nur77 nuclear export in PC12 cells is blocked by pre-treatment with leptomycin B, a nuclear export inhibitor. This pre-treatment induced a significant reduction 6-OHDA-induced cell death as demonstrated here and in another cell line (No et al., 2010). This is in accordance with results in cancer cells, where Nur77 functions as a survival factor in the nucleus and in contrast, as a potential cell death signal when migrating to the mitochondria. At this subcellular localization, it binds to Bcl-2 and converts Bcl-2 from a protector to a killer by triggering cytochrome C release and activating the caspase cascade (Zhang, 2007; Li et al., 2000; Martinou et al., 2000; Lin et al., 2004). In cancer cells, Nur77-induced apoptosis is rapid and robust. The contrasting actions of Nur77 on apoptosis are modulated by its intracellular localization which in turn is dependent upon the nature of the signal to which the cell is exposed (Zhang, 2007). Nur77 subcellular localization is controlled via nuclear localization and nuclear export signals (Hsu et al., 2004). The DNA binding domain (DBD) of Nur77 contains two nuclear localization signals whereas the ligand binding domain (LDB) contains three leucine-rich putative nuclear export signals, which are critical for exit from the nucleus. Nur77 is a direct target of various signaling cascades which can lead to its phosphorylation which in turn might impact on its activity, subcellular localization and receptor heterodimerization. Data in the literature suggest that extracellular signal-regulated kinase2 (ERK2) might be involved in Nur77-mediated cell death (Castro-Obregon et al., 2004) and that ERK1/2 is implicated in 6-OHDA neurotoxic effects in the brain (Chu et al., 2004; Kulich and Chu, 2003).

RXR is an essential heterodimerization partner for both Nur77 and Nurr1 (Maira et al., 1999; Perlmann and Jansson, 1995). High levels of the RXRγ1 isoform are found in the substantia nigra (Langlois et al., 2001; Krezel et al., 1999; Saga et al., 1999). Recent data indicate that activation of RXR receptors increases dopaminergic cell survival in models for Parkinson's disease (Friling et al., 2009; Backman et al., 2003). DHA is a polyunsaturated fatty acid that acts as an endogenous RXR ligand in the brain (Mata de Urquiza et al., 2000). It has been recently shown that a diet rich in DHA exerts a neuroprotective action against MPTP-induced toxicity by protecting against the neurotoxin-induced decrease of several dopaminergic markers into the striatum and the substantia nigra (Bousquet et al., 2007). In accordance, we show here DHA is able to prevent 6-OHDA and MPP+-induced cell death in PC12 cells by blocking Nur77 translocation from the nucleus to the cytosol. This is consistent with data in the literature indicating that RXR modulates Nur77 subcellular localization in a ligand-dependent manner. In the presence of RXR transactivation by agonists, Nur77-RXR heterodimers are formed via heterodimerization sites in their ligand-binding domains (LBDs), resulting in chromatin-binding heterodimers. This conformation silences the nuclear export signal (NES), thus maintaining nuclear localization (Zhang, 2007). In accordance with this, RXR agonists suppress apoptosis by inhibiting mitochondrial targeting (Cao et al., 2004). However, the possibility that the Nur77-RXR heterodimer complex can also be translocated outside the nucleus remains. In this case, it has been proposed that Nur77 cannot induce apoptosis due to the inability of Nur77-RXR heterodimer to interact with the mitochondria, in contrast to Nur77 alone (Kang et al., 2000).

### CONCLUSION

Nur77 is rapidly and transiently induced in midbrain dopamine neurons after the administration of 6-OHDA in vivo.

Nur77 is involved in cell death after 6-OHDA and MPTP exposure via a mechanism involving its translocation from the nucleus to the cytosol.

Transactivation of RXR by DHA silences the translocation of Nur77 to the cytosol thereby inhibiting Nur77-dependent apoptosis.

Altogether, these data suggest that Nur77 is involved in dopamine cell death via a mechanism that can be blocked by the administration of DHA. These novel findings open the way to new therapeutic strategies that may slow or stop the progression of Parkinson's disease.

### REFERENCES

Beaudry G., Langlois M.-C., Weppe I., Rouillard C. and Lévesque D. (2000) Contrasting patterns and cellular specificity of transcriptional regulation of the nuclear receptor Nerve Growth Factor-inducible B by haloperidol and clozapine in the rat forebrain. J. Neurochem. 75, 1694-1702.
Bourhis E., Maheux J., Rouillard C. and Levesque D. (2008) Extracellular signal-regulated kinases (ERK) and protein kinase C (PKC) activities are involved in the modulation of Nur77 and Nor-1 expression by dopaminergic drugs. J. Neurochem. 106, 875-888.
Bousquet M., Saint-Pierre M., Julien C., Salem N., Jr., Cicchetti F. and Calon F. (2007) Beneficial effects of dietary omega-3 polyunsaturated fatty acid on toxin-induced neuronal degeneration in an animal model of Parkinson's disease. FASEB J. 22, 1213-1225.
Cao X., Liu W., Lin F., Li H., Kolluri S. K., Lin B., Han Y. H., Dawson M. I. and Zhang X. k. (2004) Retinoid X receptor regulates Nur77/Thyroid Hormone Receptor 3-dependent apoptosis by modulating its nuclear export and mitochondrial targeting. Mol. Cell. Biol. 24, 9705-9725. Castro-Obregon S., Rao R. V., del RiO G., Chen S. F., Poksay K. S., Rabizadeh S., Vesce S., Zhang X. K., Swanson R. A. and Bredesen D. E. (2004) Alternative, nonapoptotic programmed cell death: mediation by arrestin 2, ERK2, and Nur77. J. Biol. Chem. 279, 17543-17553.
Cheng L. E., Chan F. K., Cado D. and Winoto A. (1997) Functional redundancy of the Nur77 and Nor-1 orphan steroid receptors in T-cell apoptosis. EMBO J. 16, 1865-1875.
Chiasson K., Daoust B., Levesque D. and Martinoli M. G. (2006) Dopamine D2 agonists, bromocriptine and quinpirole, increase MPP+-induced toxicity in PC12 cells. Neurotox. Res 10, 31-42.
Chu C. T., Levinthal D. J., Kulich S. M., Chalovich E. M. and DeFranco D. B. (2004) Oxidative neuronal injury. The dark side of ERK1/2. Eur. J. Biochem. 271, 2060-2066.
Crawford P. A., Sadovsky Y., Woodson K., Lee S. L. and Milbrandt J. (1995) Adrenocortical function and regulation of the steroid 21-hydroxylase gene in NGFI-B-deficient mice. Mol. Cell. Biol. 15, 4331-4316.
Darragh J., Soloaga A., Beardmore V. A., Wingate A. D., Wiggin G. R., Peggie M. and Arthur J. S. (2005) MSKs are required for the transcription of the nuclear orphan receptors Nur77, Nurr1 and Nor1 downstream of MAPK signalling. Biochem J390, 749-759.
Eells J. B., Yeung S. K. and Nikodem V. M. (2002) Dopamine neurons heterozygous for the Nurr1-null allele have reduced survival in vitro. Neurosci. Res. Commun. 30, 173-183.
Fass D. M., Butler J. E. F. and Goodman R. H. (2003) Deacetylase activity is required for cAMP activation of a subset of CREB target genes. J. Biol. Chem. 278, 43014-43019.
Friling S., Bergsland M. and Kjellander S. (2009) Activation of Retinoid X Receptor increases dopamine cell survival in models for Parkinson's disease. BMC Neurosci. 10, 146.
Gagne B., Gelinas S., Bureau G., Lagace B., Ramassamy C., Chiasson K., Valastro B. and Martinoli M. G. (2003) Effects of estradiol, phytoestrogens, and Ginkgo biloba extracts against 1-methyl-4-phenyl-pyridine-induced oxidative stress. Endocrine. 21, 89-95.
Gelinas S., Bureau G., Valastro B., Massicotte G., Cicchetti F., Chiasson K., Gagne B., Blanchet J. and Martinoli M. G. (2004) Alpha and beta estradiol protect neuronal but not native PC12 cells from paraquat-induced oxidative stress. Neurotox. Res 6, 141-148.
Gelinas S, and Martinoli M. G. (2002) Neuroprotective effect of estradiol and phytoestrogens on MPP+-induced cytotoxicity in neuronal PC12 cells. J Neurosci Res 70, 90-96.
Gibrat C., Bousquet M., Saint-Pierre M., Levesque D., Calon F., Rouillard C. and Cicchetti F. (2010) Cystamine prevents MPTP-induced toxicity in young adult mice via the up-regulation of the brain-derived neurotrophic factor. Prog. Neuro-psychopharmacol. Biol. Psychiat. 34, 193-203.
Glaser J. R. and Glaser E. M. (2000) Stereology, morphometry, and mapping: the whole is greater than the sum of its parts. J Chem. Neuroanat. 20, 115-126.
Heatwole V. M. (1999) TUNEL assay for apoptotic cells. Methods Mol. Biol. 115, 141-148.
Hirata Y., Meguro T. and Kiuchi K. (2006) Differential effect of nerve growth factor on dopaminergic neurotoxin-induced apoptosis. J Neurochem 99, 416-425.
Hsu H. C., Zhou T. and Mountz J. D. (2004) Nur77 family of nuclear hormone receptors. Curr. Drug Targets. Inflamm. Allergy 3, 413-423.
Jiang C., Wan X., He Y., Pan T., Jankovic J. and Le W. (2005) Age-dependent dopaminergic dysfunction in Nurr1 knockout mice. Exp. Neurol. 191, 154-162.
Kadkhodaei B., Ito T., Joodmardi E., Mattsson B., Rouillard C., Carta M., Muramatsu S. I., Sumi-Ichinose C., Nomura T., Metzger D., Chambon P., Lindqvist E., Larsson N. G., Olson L., Bjorklund A., Ichinose H. and Perlmann T. (2009) Nurr1 Is required for maintenance of maturing and adult midbrain dopamine neurons. J. Neurosci. 29, 15923-15932.
Kang H. J., Song M. R., Lee S. K., Shin E. C., Choi Y. H., Kim S. J., Lee J. W. and Lee M. O. (2000) Retinoic acid and its receptors repress the expression and transactivation functions of Nur77: a possible mechanism for the inhibition of apoptosis by retinoic acid. Exp Cell Res 256, 545-554.
Kovalovsky D., Refojo D., Liberman A. C., Hochbaum D., Pereda M. P., Coso O. A., Stalla G. K., Holsboer F. and Artz E. (2002) Activation and induction of NUR77/NURR1 in corticotrophs by CRH/cAMP: involvement of calcium, protein kinase A, and MPAK pathways. Mol. Endocrinol. 16, 1638-1651.
Krezel W., Kastner P. and Chambon P. (1999) Differential expression of retinoid receptors in the adult mouse central nervous system. Neuroscience 89, 1291-1300.
Kulich S. M. and Chu C. T. (2003) Role of reactive oxygen species in extracellular signal-regulated protein kinase phosphorylation and 6-hydroxydopamine cytotoxicity. J Biosci. 28, 83-89.
Langlois M.-C., Beaudry G., Rouillard C. and Levesque D. (2001) Impact of neuroleptic administration on coordinate expression of nuclear receptors in the cortex and striatal complex of the rat brain. Neurosci. 106, 117-128.
Le W., Conneely O. M., He Y., Jankovic J. and Appel S. H. (1999) Reduced Nurr1 expression increases the vulnerability of mesencephalic dopamine neurons to MPTP-induced injury. J. Neurochem. 73, 2218-2221.
Le W. D., Xu P., Jankovic J., Jiang H., Appel S. H., Smith R. G. and Vassilatis D. K. (2003) Mutations in NR4A2 associated with familial Parkinson disease. Nat. Genet. 33, 85-89.
Lee S. L., Wesselschmidt R. L., Linette G. P., Kanagawa O., Russell J. H. and Milbrandt J. (1995) Unimpaired thymic and peripheral T cell death in mice lacking the nuclear receptor NGFI-B (Nur77). Science 269, 532-535.
Levesque D. and Rouillard C. (2007) Nur77 and Retinoid X receptors: critical factors in dopamine-related neuroadaptation. Trends in Neurosci. 30, 22-30.
Li H., Kolluri S. K., Gu J., Dawson M. I., Cao X., Hobbs P. D., Lin B., Chen G., Lu J., Lin F., Xie Z., Fontana J. A., Reed J. C. and Zhang X. (2000) Cytochrome c release and apoptosis induced by mitochondrial targeting of nuclear orphan receptor TR3. Science 289, 1159-1164.
Lin B., Kolluri S. K., Lin F., Liu W., Han Y. H., Cao X., Dawson M. I., Reed J. C. and Zhang X. K. (2004) Conversion of Bcl-2 from protector to killer by interaction with nuclear orphan receptor Nur77/TR3. Cell 116, 527-540.
Maheux J., Ethier I., Rouillard C. and Levesque D. (2005) Induction patterns of transcription factors of the Nur family (Nurr1, Nur77 and Nor-1) by typical and atypical antipsychotics in the mouse brain: Implication for their mechanism of action. J. Pharmacol. Exp. Ther. 313, 460-473.
Maira M., Martens C., Philips A. and Drouin J. (1999) Heterodimerization between members of the Nur subfamily of orphan nuclear receptors as a novel mechanism for gene activation. Mol. Cell. Biol. 19, 7549-7557.
Martinou J. C., Desagher S, and Antonsson B. (2000) Cytochrome c release from mitochondria: all or nothing Nat. Cell Biol. 2, E41-E43. Mata de Urquiza A., Liu S., Sjoberg M., Zetterstrom R. H., Griffiths W., Sjovall J. and Perlmann T. (2000) Docosahexaenoic acid, a ligand for the retinoid X receptor in mouse brain. Science 290, 2140-2144.
Maxwell M. A. and Muscat G. E. O. (2006) The NR4A subgroup. immediate early response genes with pleiotropic physiological roles. Nuclear Receptor Signaling 4, e002—Epub 2006 Feb. 8.
No H., Bang Y., Lim J. H., Kim S. S., Choi H. S, and Choi H. J. (2010) Involvement of induction and mitochondrial targeting of orphan nuclear receptor Nur77 in 6-OHDA-induced SH-SY5Y cell death. Neurochem. Int. 56, 620-626.
Paxinos G. and Watson C. (1986) The Rat Brain in Stereotaxic Coordinates. Academic Press, San Diego.
Perlmann T. and Jansson L. (1995) A novel pathway for vitamin A signaling mediated by RXR heterodimerization with NGFI-B and NURR1. Genes Dev. 9, 769-782.
Roehm N. W., Rodgers G. H., Hatfield S. M. and Glasebrook A. L. (1991) An improved colorimetric assay for cell proliferation and viability utilizing the tetrazolium salt XTT. J Immunol. Methods 142, 257-265.
Rousseau J., Cossette L., Grenier S, and Martinoli M. G. (2002) Modulation of prolactin expression by xenoestrogens. Gen. Comp Endocrinol 126, 175-182.
Saga Y., Kobayashi M., Ohta H., Murai N., Nakai N., Oshima M. and Taketo M. M. (1999) Impaired extrapyramidal function caused by the targeted disruption of retinoid X receptor RXRgammal isoform. Genes Cells 4, 219-228.
Schmued L. C., Albertson C. and Slikker W., Jr. (1997) Fluoro-Jade: a novel fluorochrome for the sensitive and reliable histochemical localization of neuronal degeneration. Brain Res 751, 37-46.
Schmued L. C. and Hopkins K. J. (2000) Fluoro-Jade B: a high affinity fluorescent marker for the localization of neuronal degeneration. Brain Res. 874, 123-130.
Suzuki S., Suzuki N., Mirtsos C., Horacek T., Lye E., Noh S. K., Ho A., Bouchard D., Mak T. W. and Yeh W. C. (2003) Nur77 as a survival factor in tumor necrosis factor signaling. Proc. Natl. Acad. Sci. USA 100, 8276-8280.
Tremblay M.-É., Saint-Pierre M., Bourhis E., Levesque D., Rouillard C. and Cicchetti F. (2006) Neuroprotective effects of cystamine in aged parkinsonian mice. Neurobiol. Aging 27, 862-870.
Wallen-Mackenzie A., Mata de Urquiza A., Petersson S., Rodriguez F. J., Friling S., Wagner J., Ordentlich P., Lengqvist J., Heyman R. A., Arenas E. and Perlmann T. (2003) Nurr1-RXR heterodimers mediate RXR ligand-induced signaling in neuronal cells. Genes Dev. 17, 3036-3047.
Yamamoto N., Sawada H., Izumi Y., Kume T., Katsuki H., Shimohama S, and Akaike A. (2007) Proteasome inhibition induces glutathione synthesis and protects cells from oxidative stress: relevance to Parkinson disease. J. Biol. Chem. 282, 4364-4372.
Zhang X. k. (2007) Targeting Nur77 translocation. Expert Opin. Ther. Targets 11, 69-79.
Zuch C. L., Nordstroem V. K., Briedrick L. A., Hoernig G. R., Granholm A. C. and Bickford P. C. (2000) Time course of degenerative alterations in nigral dopaminergic neurons following a 6-hydroxydopamine lesion. J Comp Neurol 427, 440-454.

1. A method for identifying a compound capable of preventing or inhibiting dopaminergic cell-death, said method comprising the steps of assessing the ability of a compound to achieve at least one of:
a) inhibit or prevent the expression of Nur77 in the nucleus of a nigral dopaminergic neuron;
b) inhibit or prevent the translocation of Nur77 to the cytosol of a nigral dopaminergic neuron;
c) increase or promote the transactivation of RXR in the nucleus of a nigral dopaminergic neuron; or
d) increase or promote the formation of RXR-Nur77 heterodimers in the nucleus of a nigral dopaminergic neuron;
whereby the ability of said compound to achieve at least one of said goal listed in a) to d) is an indication that said compound is a potential drug for preventing or inhibiting dopaminergic cell-death.

2. The method of claim 1, wherein said expression of Nur77 in step a) is mRNA expression or protein expression.

3. The method of claim 1, comprising the steps of:
contacting a candidate compound with Nur-77 or a Nur77-producing cell; and
evaluating the ability of said compound to bind to Nur-77;
whereby the ability of said compound to bind Nur77 is an indication that said compound is a potential drug for preventing or inhibiting dopaminergic cell-death.

4. The method of claim 1, comprising the steps of:
contacting a candidate compound with Nur-77 or a Nur77-producing cell; and
evaluating the ability of said compound to inhibit Nur-77 dependent apoptosis,
whereby the ability of said compound to inhibit Nur77-dependent apoptosis is an indication that said compound is a potential drug for preventing/protecting dopaminergic cell-death.

5. The method of claim 4, wherein the ability of said compound to inhibit Nur77-dependent apoptosis is evaluated by assessing the nuclear translocation of Nur77 in a dopaminergic cell.

6. The method of claim 1, comprising the steps of:
contacting a candidate compound with Retinoid X Receptor (RXR) or a RXR-presenting-cell; and
evaluating the ability of said compound to bind to RXR or a RXR-presenting cell;
whereby the ability of said compound to bind to RXR is an indication that said compound is a potential drug for preventing or inhibiting dopaminergic cell-death.

7. The method of claim 1, comprising the steps of:
a) contacting a candidate compound with Retinoid X Receptor (RXR) or a RXR-presenting cell; and
b) evaluating the ability of said compound to form a transcriptional complex with RXR (RXR-cpd);
whereby the ability of said compound to form a transcriptional complex with RXR is an indication that said compound is a potential drug for preventing or inhibiting dopaminergic cell-death.

8. The method of claim 7, further comprising the step of:
c) assessing the ability of said transcriptional complex to inhibit the translocation of Nur77 to the cytosol of said dopaminergic cell;
whereby the ability of said complex to inhibit Nur77 translocation is an indication that said compound is a potential drug for preventing or inhibiting dopaminergic cell-death.

9. The method of claim 1, wherein the steps are carried out in vitro using a recombinant protein system.

10. The method of claims 1, wherein the steps are carried out in cells that express RXR and Nur77.

11. The method of claim 1, wherein the cell expresses the Nur77 or RXR proteins constitutively or transiently.

12. The method of claim 11, wherein the cell is a dopaminergic neurons in culture or a PC12 cell.

13. The method of claim 12, wherein the cell is a PC12 cell.

14. A method for protecting against or preventing dopaminergic cell-death comprising the step of inhibiting Nur77-dependent apoptosis.

15. The method of claim 14, wherein said inhibiting Nur77-dependent apoptosis is carried out by at least one of:
a) inhibiting or preventing the expression of Nur77 in the nucleus of nigral dopamine neurons;
b) inhibiting or preventing the translocation of Nur77 to the cytosol of nigral dopamine neurons;
c) increasing or promoting the transactivation of RXR in the nucleus of nigral dopaminergic neurons; or
d) increasing or promoting the formation of RXR-Nur77 heterodimers in the nucleus of nigral dopaminergic neurons.

16. A method for the prevention or treatment of Parkinson's disease in a subject comprising administering to the human a pharmaceutically effective amount of a compound inhibiting Nur77-dependent apoptosis as defined in claim 1.

17. The method of claim 16, wherein the compound inhibiting Nur-77 dependent apoptosis is a Retinoid X Receptor (RXR) ligand.

18. The method of claim 17, wherein the RXR ligand induces the transactivation of the RXR in the nucleus of nigral dopamine neurons inducing the formation of a RXR-Nur77 complex.

19. The method of claim 17, wherein the RXR ligand blocks the translocation of the RXR-Nur77 complex from the nucleus to the cytosol of nigral dopamine neurons.

20. The method of claim 16, wherein the subject is a human.

21. The method of claim 20, wherein the treatment may be administered before or after symptoms appear in the human.

22. The method of claim 17, wherein the RXR ligand is docosahexaenoic acid (DHA) or its metabolites or derivatives.

23. The method of claim 22, wherein the RXR ligand is docosahexaenoic acid (DHA).
